(19) United States
(12) Patent Application Publication
(10) Pub. No.: US 2009/0258953 A1
(43) Pub. Date: Oct. 15, 2009

(54) USE OF VINYL ACETATE-SULFONATE COPOLYMERS AS SOLUBILIZERS FOR SLIGHTLY WATER-SOLUBABLE COMPOUNDS

(75) Inventors: Rainer Dobrawa, Mannheim (DE); Nathalie Bouillo, Baden-Baden (DE); Ronald Frans Maria Lange, Ludwigshafen (DE); Kathrin Meyer-Böhm, Feucht (DE); Murat Mertoglu, Ludwigshafen (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/303,509

(22) PCT Filed: May 31, 2007
(86) PCT No.: PCT/EP2007/055309

§ 371 Date: Dec. 4, 2008

(30) Foreign Application Priority Data
Jun. 7, 2006 (EP) 06115089.2

(51) Int. Cl. A61K 47/32; C08F 220/18

(52) U.S. Cl. 514/772.6; 526/329.5

(57) ABSTRACT

The use of copolymers obtainable by free radical-initiated copolymerization of
a) 80 to 99.5% by weight of vinyl acetate and
b) 0.5 to 20% by weight of a monoolefinically unsaturated monomer having sulfonate groups
   as solubilizers for slightly water-soluble substances.

The invention relates to the use of vinyl acetate-sulfonate copolymers as solubilizers of slightly water-soluble bioactive substances. The invention further relates to corresponding preparations for use in humans, animals and plants, and to specific copolymers.

The solubilization of hydrophobic, i.e. slightly water-soluble substances has acquired very great practical importance in the production of homogeneous preparations of bioactive substances.

Solubilization means the rendering soluble of substances which are slightly soluble or insoluble in a particular solvent, especially water, by surface-active compounds, the solubilizers. Such solubilizers are able to convert poorly water-soluble or water-insoluble substances into clear, at the most opalescent aqueous solutions without the chemical structure of these substances undergoing an alteration thereby (cf. Römpp Chemie Lexikon, 9th edition, Vol. 5, p. 4203. Thieme Verlag, Stuttgart, 1992).

The produced solubilisates are characterized by the poorly water-soluble or water-insoluble substance being in the form of a colloidal solution in the aggregates of molecules of the surface-active compounds which form in aqueous solution—for example the so-called micelles. The resulting solutions are stable single-phase systems which appear optically clear or opalescent and can be produced without energy input.

Solubilizers may for example improve the appearance of cosmetic formulations and of fluid preparations by making the formulations transparent. In the case of pharmaceutical preparations, there may additionally be an increase in the bioavailability and thus the effect of drugs through the use of solubilizers.

The solubilizers employed for pharmaceutical drugs and cosmetic active substances are mainly surfactants such as ethoxylated (hydrogenated) castor oil, ethoxylated sorbitan fatty acid esters or ethoxylated hydroxystearic acid. However, the solubilizers described above and employed to date show a number of technical disadvantages when used.

The solubilizing effect of known solubilizers for some slightly soluble drugs such as, for example, clotrimazol is only low.

In addition, solubilizers disclosed to date are usually liquid or semisolid compounds which exhibit less favorable processing properties because of this.

Vinyl acetate-sulfonate copolymers are known per se. Corresponding copolymers and their use as textile finishes are described for example in U.S. Pat. No. 2,834,759.

JP-A 51003383 discloses copolymers of vinyl acetate and allylsulfonates and their use as emulsifiers for epoxy resins.

JP-A 50160334 discloses copolymers of vinyl acetate and allylsulfonates and their use as emulsifiers for acrylic resin coating compositions.

JP-A 09202812 discloses the preparation of polyvinyl esters in the presence of small amounts of allylsulfonates, the allylsulfonates being known as phase-transfer agents.

GB 1350282 discloses the use of copolymers of vinyl acetate and small amounts of unsaturated sulfonates as coating agents.

The object was to provide novel solubilizers for pharmaceutical, cosmetic, food industry and agrotechnical applications which are able to act in aqueous medium as solubilizers for appropriate slightly water-soluble active ingredients.

The object has been achieved according to the invention through the use of vinyl acetate-sulfonate copolymers as solubilizers for slightly water-soluble substances.

Vinyl acetate-sulfonate copolymers are defined according to the invention as copolymers of vinyl acetate and monoethylenically unsaturated monomers comprising sulfonate groups and, if appropriate, further comonomers.

Monomers comprising sulfonate groups which are suitable according to the invention are monoethylenically unsaturated sulfonic acid compounds. Suitable sulfonic acid compounds are for example alkyl sulfonates of acrylic acid or of methacrylic acid such as sulfopropyl acrylate or sulfopropyl methacrylate. Likewise suitable are linear or branched C₁-C₁₀-alkylsulfonamides of acrylic acid or of methacrylic acid such as, for example, acrylamidomethylpropanesulfonic acid (AMPS). Also suitable are w-alkene-1-sulfonic acids having 2 to 10 C atoms.

Preferred monomers are selected from the group consisting of vinylsulfonate, allylsulfonate, methallylsulfonate (2-methyl-2-propenesulfonate), sulfopropyl acrylate and sulfopropyl methacrylate.

The sulfonate monomers are normally employed in the form of their salts. Particularly suitable salts are the alkali metal salts, for example lithium, potassium or sodium salts, with preference for sodium salts and potassium salts. The sulfonate monomers are normally fed into the polymerization in the form of aqueous solutions, with the concentration of the sulfonate monomer possibly being 20 to 70% by weight, preferably 20 to 40% by weight.

In an alternative embodiment, the sulfonic acid-containing monomers can also be employed as solids and be dissolved or suspended in the solvent. The monomer slowly dissolves during the polymerization.

The copolymers having sulfonate groups are obtainable by copolymerization of a) 80 to 99.5% by weight of vinyl acetate and b) 0.5 to 20% by weight of the sulfonate monomer. It is preferred to employ a) 85 to 98% by weight and b) 2 to 15% by weight, particularly preferably a) 85 to 95% by weight and b) 5 to 15% by weight.

General processes for preparing vinyl acetate-sulfonate copolymers are known per se. The preparation takes place as polymerization initiated by free radicals using water-miscible organic solvents. Examples of suitable solvents are alcohols such as methanol, ethanol, n-propanol and isopropanol, and glycols such as ethylene glycol and glycerol. Also suitable as solvents are acetic esters such as, for example, ethyl acetate or butyl acetate. The preferred solvent is n-propanol.

The polymerization is preferably carried out at temperatures from 60 to 100° C. The polymerization can be carried out under atmospheric pressure or under a superatmospheric pressure of up to 1.5 MPa, with preference for atmospheric pressure.

The polymerization is initiated by employing free-radical initiators. The amounts of initiator or initiator mixtures used based on monomer employed are between 0.01 and 10% by weight, preferably between 0.3 and 5% by weight.

In a preferred embodiment of the invention, the polymerization is carried out in such a way that initially an oil-soluble free-radical initiator, meaning according to the invention a free-radical initiator which is soluble in organic solvents and insoluble in water, is employed, and a water-soluble free-radical initiator is employed at the start of the after-polymerization. The phase of after-polymerization starts in the feed process after the monomer feeds have been completely added. In the batch process, the phase of after-polymerization is generally considered to start after two thirds of the total reaction time.

Suitable free-radical initiators are both organic and inorganic peroxides such as sodium persulfate or azo initiators such as 2,2′-azobis[2-(2-imidazolin-2-yl)propane] dihydrochloride, 2,2′-azobis(2-methylpropionamide) dihydrochloride), 2,2′-azobis(2-aminidinopropane) dihydrochloride, 2,2′-azobis(2-methylbutyronitrile), 2,2′-azobis[2-(2-imidazolin-2-yl)propane], 2,2′-azobis(2-methylbutyronitrile), 2,2′-azobisisobutyronitrile, 2,2′-azobis(2,4-dimethylvaleronitrile), dimethyl 2,2′-azobis(2-methylpropionate), 2,2′-azobis(4-methoxy-2,4-dimethylvaleronitrile).

Examples of peroxide initiators are dibenzoyl peroxide, diacetyl peroxide, succinyl peroxide, tert-butyl perpivalate, tert-butyl 2-ethylhexanoate, tert-butyl perneodecanoate, tert-butyl permaleate, bis(tert-butylperoxy)cyclohexane, tert-butylperoxy isopropyl carbonate, tert-butyl peracetate, 2,2-bis(tert-butylperoxy)butane, dicumyl peroxide, di-tert-amyl peroxide, di-tert-butyl peroxide, p-menthane hydroperoxide, pinane hydroperoxide, cumene hydroperoxide, tert-butyl hydroperoxide, hydrogen peroxide and mixtures of said initiators. Said initiators may also be used in combination with redox components such as ascorbic acid.

The use of solvent-soluble (and thus poorly water-soluble) free-radical initiators during the polymerization is particularly preferred. Examples of preferred solvent-soluble free-radical initiators are 2,2′-azobisisobutyronitrile, 2,2′-azobis(2-methylbutyronitrile), 2,2′-azobis(2,4-dimethylvaleronitrile), dimethyl 2,2′-azobis(2-methylpropionate), 2,2′-azobis(4-methoxy-2,4-dimethylvaleronitrile), dibenzoyl peroxide, diacetyl peroxide, succinyl peroxide, tert-butyl perpivalate, tert-butyl 2-ethylhexanoate, tert-butyl perneodecanoate, tert-butyl permaleate, bis(tert-butylperoxy)cyclohexane, tert-butylperoxy isopropyl carbonate, tert-butyl peracetate, 2,2-bis(tert-butylperoxy)butane, dicumyl peroxide, di-tert-amyl peroxide, di-tert-butyl peroxide, p-menthane hydroperoxide, pinane hydroperoxide, cumene hydroperoxide.

The free-radical polymerization may if appropriate place in the presence of emulsifiers, if appropriate further protective colloids, if appropriate buffer systems and if appropriate subsequent pH adjustment using bases or acids.

Suitable molecular weight regulators are sulfhydryl compounds such as alkyl mercaptans, e.g. n-dodecyl mercaptan, tert-dodecyl mercaptan, thioglycolic acid and esters thereof, mercaptoalkanols such as mercaptoethanol. Further suitable regulators are mentioned for example in DE 197 12 247 A1, page 4. The necessary amount of the molecular weight regulators is in the range from 0 to 5% by weight based on the amount of monomers to be polymerized, in particular 0.05 to 2% by weight, particularly preferably 0.1 to 1.5% by weight. Mercaptoethanol is preferably employed.

The copolymers which can be used according to the invention can also be prepared in the presence of suitable difunctional crosslinker components (crosslinkers) and/or in the presence of suitable regulators.

Suitable crosslinkers are monomers having a crosslinking function, for example compounds having at least two ethylenically unsaturated, nonconjugated double bonds in the molecule.

Examples thereof are acrylic esters, methacrylic esters, allyl ethers or vinyl ethers of at least dihydric alcohols. The OH groups of the underlying alcohols may moreover be wholly or partly etherified or esterified; however, the crosslinkers comprise at least two ethylenically unsaturated groups.

Examples of underlying alcohols are dihydric alcohols such as 1,2-ethane-diol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic acid neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000. Apart from the homopolymers of ethylene oxide and propylene oxide it is also possible to employ block copolymers of ethylene oxide or propylene oxide or copolymers which comprise incorporated ethylene oxide and propylene oxide groups. Examples of underlying alcohols having more than two OH groups are trimethylolpropane, glycerol, pentaerythritol, 1,2,5-pentanetriol, 1,2,6-hexanetriol, triethoxycyanuric acid, sorbitan, sugars such as sucrose, glucose, mannose. The polyhydric alcohols can, of course, also be employed as the corresponding ethoxylates or propoxylates after reaction with ethylene oxide or propylene oxide. The polyhydric alcohols can also initially be converted into the corresponding glycidyl ethers by reaction with epichlorohydrin.

Further suitable crosslinkers are the vinyl esters or the esters of monohydric unsaturated alcohols with ethylenically unsaturated C₃ to C₆ carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-g-octadecen-1-ol. The monohydric unsaturated alcohols can, however, also be esterified with polybasic carboxylic acids, for example malonic acid, tartaric acid, trimellitic acid, phthalic acid, terephthalic acid, citric acid or succinic acid.

Further suitable crosslinkers are esters of unsaturated carboxylic acids with the polyhydric alcohols described above, for example of oleic acid, crotonic acid, cinnamic acid or 10-undecenoic acid.

Suitable crosslinkers are additionally straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of 200 to 20 000.

Further suitable crosslinkers are the acrylamides, methacrylamides and N-allylamines of at least difunctional amines. Examples of such amines are 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides from allylamine and unsaturated carboxylic acids such as acrylic acid, methacrylic acid, itaconic acid, maleic acid, or at least dibasic carboxylic acids as have been described above.

Further suitable crosslinkers are triallylamine and triallylmonoalkylammonium salts, e.g. triallylmethylammonium chloride or -ammonium methyl sulfate.

Also suitable are N-vinyl compounds of urea derivatives, at least difunctional amides, cyanurates or urethanes, for example of urea, ethyleneurea, propyleneurea or tartramide, e.g. N,N′-divinylethyleneurea or N,N′-divinylpropyleneurea.

Further suitable crosslinkers are divinyldioxane, tetraallylsilane or tetravinylsilane.

It is, of course, also possible to employ mixtures of the aforementioned compounds. The crosslinkers preferably employed are those soluble in the monomer mixture.

The monomer or a monomer mixture or the emulsion of monomer(s) are introduced together with the initiator, which is usually present in solution, into a stirred reactor at the polymerization temperature (batch process), or if appropriate metered continuously or in a plurality of consecutive stages into the polymerization reactor (feed process). It is usual in the feed process for the reactor to be charged before the actual polymerization starts, besides which the organic solvent or water (in order to make stirring of the reactor possible), already with partial quantities, rarely the whole quantity intended for the polymerization, of the starting materials such as emulsifiers, protective colloids, monomers, regulators etc. or partial quantities of the feeds (generally monomer or emulsion feed and initiator feed).

The copolymers obtained in this way are water-soluble or water-dispersible. The copolymers may have Fikentscher K values, measured as 1% by weight solution in water, of from 4 to 30, preferably 5 to 25, particularly preferably 5 to 15.

### Applications:

The copolymers to be used according to the invention can be employed in principle in all areas where substances of only low or zero solubility in water are either intended to be employed in aqueous preparations or intended to display their effect in aqueous medium. The copolymers are accordingly used as solubilizers for slightly water-soluble substances, in particular bioactive substances.

The term “slightly water-soluble” includes according to the invention also practically insoluble substances and means that at least 30 to 100 g of water are required per g of substance for the substance to dissolved in water at 20° C. In the case of practically insoluble substances, at least 10 000 g of water are required per g of substance.

In the context of the present invention, slightly water-soluble bioactive substances mean active pharmaceutical ingredients for humans and animals, cosmetic or agrochemical active substances or dietary supplements or dietetic active substances.

Further slightly soluble substances suitable for solubilization are also colorants such as inorganic or organic pigments.

The present invention provides in particular amphiphilic compounds for use as solubilizers for pharmaceutical and cosmetic preparations and for food preparations. They have the property of solubilizing slightly soluble active ingredients in the area of pharmacy and cosmetics, slightly soluble dietary supplements, for example vitamins and carotenoids, but also slightly soluble active substances for use in crop protection agents and veterinary medical active ingredients.

### Solubilizers for Cosmetics:

The copolymers can be employed according to the invention as solubilizers in cosmetic formulations. They are suitable for example as solubilizers for cosmetic oils. They have a good solubilizing capacity for fats and oils such as peanut oil, jojoba oil, coconut oil, almond oil, olive oil, palm oil, castor oil, soybean oil or wheatgerm oil or for essential oils such as dwarf pine oil, lavender oil, rosemary oil, spruce needle oil, pine needle oil, eucalyptus oil, peppermint oil, sage oil, bergamot oil, terpentine oil, melissa oil, juniper oil, lemon oil, anise oil, cardamom oil, camphor oil etc. or for mixtures of these oils.

The polymers of the invention can further be used as solubilizers for UV absorbers which are slightly soluble or insoluble in water, such as, for example, 2-hydroxy-4-methoxybenzophenone (Uvinul® M 40, from BASF), 2,2′,4,4′-tetrahydroxy-benzophenone (Uvinul® D 50), 2,2′-dihydroxy-4,4′-dimethoxybenzophenone (Uvinul®D49), 2,4-dihydroxybenzophenone (Uvinul® 400), 2′-ethylhexyl 2-cyano-3,3-diphenylacrylate (Uvinul® N 539), 2,4,6-trianilino-p-(carbo-2′-ethylhexyl-1′-oxy)-1,3,5-triazine (Uvinul® T 150), 3-(4-methoxybenzylidene)camphor (Eusolex® 6300, from Merck), 2-ethylhexyl N,N-dimethyl-4-aminobenzoate (Eusolex® 6007), 3,3,5-trimethylcyclohexyl salicylate, 4-isopropyldibenzoylmethane (Eusolex® 8020), 2-ethylhexyl p-methoxycinnamate and 2-isoamyl p-methoxycinnamate, and mixtures thereof.

The present invention therefore also relates to cosmetic preparations which comprise at least one of the copolymers of the invention having the composition stated at the outset as solubilizers. Preferred preparations are those which, besides the solubilizer, comprise one or more slightly soluble cosmetic active substances, for example the abovementioned oils or UV absorbers.

These formulations are water- or water/alcohol-based solubilizates. The solubilizers of the invention are employed in the ratio of from 0.2:1 to 20:1, preferably 1:1 to 15:1, particularly preferably 2:1 to 12:1, to the slightly soluble cosmetic active substance.

The content of solubilizer of the invention in the cosmetic preparation is in the range from 1 to 50% by weight, preferably 3 to 40% by weight, particularly preferably 5 to 30% by weight, depending on the active substance.

It is possible in addition for further auxiliaries to be added to this formulation, for example nonionic, cationic or anionic surfactants such as alkyl polyglycosides, fatty alcohol sulfates, fatty alcohol ether sulfates, alkanesulfonates, fatty alcohol ethoxylates, fatty alcohol phosphates, alkylbetaines, sorbitan esters, POE-sorbitan esters, sugar fatty acid esters, fatty acid polyglycerol esters, fatty acid partial glycerides, fatty acid carboxylates, fatty alcohol sulfosuccinates, fatty acid sarcosinates, fatty acid isethionates, fatty acid taurinates, citric acid esters, silicone copolymers, fatty acid polyglycol esters, fatty acid amides, fatty acid alkanolamides, quaternary ammonium compounds, alkylphenol ethoxylates, fatty amino ethoxylates, cosolvents such as ethylene glycol, propylene glycol, glycerol and others.

Further ingredients which may be added are natural or synthetic compounds, e.g. lanolin derivatives, cholesterol derivatives, isopropyl myristate, isopropyl palmitate, electrolytes, colorants, preservatives, acids (e.g. lactic acid, citric acid).

These formulations are used for example in bath additives such as bath oils, aftershaves, face tonics, hair tonics, eau de cologne, eau de toilette and in sunscreen compositions. A further area of use is the oral care sector, for example in mouthwashes, toothpastes, denture adhesive creams and the like.

The copolymers are also suitable for industrial applications for example for preparations of slightly soluble coloring agents, in toners, preparations of magnetic pigments and the like.

### Description of the Solubilization Method:

The copolymers of the invention can be employed for preparing solubilizates for cosmetic formulations either as 100% pure substance or, preferably, as aqueous solution.

Normally, the solubilizer will be dissolved in water and vigorously mixed with the slightly soluble cosmetic active substance to be used in each case.

However, it is also possible for the solubilizer to be mixed vigorously with the slightly soluble cosmetic active substance to be used in each case and then for demineralized water to be added while stirring continuously.

### Solubilizers for Pharmaceutical Applications:

The claimed copolymers are likewise suitable for use as solubilizer in pharmaceutical preparations of any type which may comprise one or more drugs which are slightly soluble or insoluble in water, and vitamins and/or carotenoids. Aqueous solutions or solubilizates for oral administration are of particular interest in this connection. Thus, the claimed copolymers are suitable for use in oral dosage forms such as tablets, capsules, powders, solutions. In these they may increase the bioavailability of the slightly soluble drug. Solid solutions of active ingredient and solubilizer are used in particular.

It is possible to employ for parenteral administration besides solubilizers also emulsions, for example fatty emulsions. The claimed copolymers are also suitable for processing a slightly soluble drug for this purpose.

Pharmaceutical formulations of the abovementioned type can be obtained by processing the claimed copolymers with active pharmaceutical ingredients by conventional methods and with use of known and novel active ingredients.

The application of the invention may additionally comprise pharmaceutical excipients and/or diluents. Excipients which are particularly mentioned are cosolvents, stabilizers, preservatives.

The active pharmaceutical ingredients used are insoluble or sparingly soluble in water. According to DAB 9 (German Pharmacopeia), the solubility of active pharmaceutical ingredients is categorized as follows: sparingly soluble (soluble in 30 to 100 parts of solvent); slightly soluble (soluble in 100 to 1000 parts of solvent); practically insoluble (soluble in more than 10 000 parts of solvent). The active ingredients may in this connection come from any range of indications.

Examples which may be mentioned here are benzodiazepines, antihypertensives, vitamins, cytostatics—especially Taxol, anesthetics, neuroleptics, antidepressants, antibiotics, antimycotics, fungicides, chemotherapeutics, urologicals, platelet aggregation inhibitors, sulfonamides, spasmolytics, hormones, immunoglobulins, sera, thyroid therapeutics, psychoactive drugs, antiparkinson agents and other antihyperkinetics, opthalmologicals, neuropathy products, calcium metabolism regulators, muscle relaxants, anesthetics, lipid-lowering agents, hepatotherapeutics, coronary agents, cardiac agents, immunotherapeutics, regulatory peptides and their inhibitors, hypnotics, sedatives, gynecologicals, gout remedies, fibrinolytics, enzyme products and transport proteins, enzyme inhibitors, emetics, blood flow stimulators, diuretics, diagnostic aids, corticoids, cholinergics, biliary therapeutics, anti asthmatics, bronchodilators, beta-receptor blockers, calcium antagonists, ACE inhibitors, arteriosclerosis remedies, antiinflammatory drugs, anticoagulants, antihypertensives, antihypoglycemics, antihypertensives, antifibrinolytics, antiepileptics, antiemetics, antidotes, antidiabetics, antiarrhythmics, antianemics, antiallergics, anthelmintics, analgesics, analeptics, aldosterone antagonists, slimming agents.

One possible production variant is to dissolve the solubilizer in the aqueous phase, if appropriate with gentle heating, and subsequently to dissolve the active ingredient in the aqueous solubilizer solution. It is likewise possible to dissolve solubilizer and active ingredient simultaneously in the aqueous phase.

It is also possible to use the copolymers of the invention as solubilizer for example by dispersing the active ingredient in the solubilizer, if appropriate with heating, and mixing with water while stirring.

A further possibility is for the solubilizers to be processed in the melt with the active ingredients. It is possible in this way in particular to obtain solid solutions. Also suitable for this purpose is the melt extrusion process, inter alia. A further possibility for producing solid solutions is also to prepare solutions of solubilizer and active ingredient in suitable organic solvents and subsequently to remove the solvent by usual processes. Also suitable for producing solid solutions ase injection molding processes and melt granulation processes.

The invention therefore also relates in general to pharmaceutical preparations which comprise at least one of the copolymers of the invention as solubilizer. Preferred preparations are those which, besides the solubilizer, comprise an active pharmaceutical ingredient which is slightly soluble or insoluble in water, for example from the abovementioned areas of indication.

Particularly preferred pharmaceutical preparations from those mentioned above are formulations which can be administered orally.

The content of solubilizer of the invention in the pharmaceutical preparation is in the range from 1 to 75% by weight, preferably 5 to 60% by weight, particularly preferably 5 to 50% by weight, depending on the active ingredient.

A further particularly preferred embodiment relates to pharmaceutical preparations in which the active ingredients and the solubilizer are present as solid solution. In this case, the ratio of solubilizers to active ingredient is preferably from 1:1 to 4:1 by weight.

### Solubilizers for Food Preparations:

Besides use in cosmetics and pharmacy, the copolymers of the invention are also suitable as solubilizers in the food sector for nutritional substances, auxiliaries or additives which are slightly soluble or insoluble in water, such as, for example, fat-soluble vitamins or carotenoids. Examples which may be mentioned are beverages colored with carotenoids.

### Solubilizers for Crop Protection Preparations:

Use of the copolymers of the invention as solubilizers in agrochemistry may comprise inter alia formulations which comprise pesticides, herbicides, fungicides or insecticides, especially including preparations of crop protection agents employed as formulations for spraying or watering.

The sulfonate copolymers of the invention are distinguished by a particularly good solubilizing effect.

The preparation and use of the copolymers of the invention is explained in more detail in the following examples.

### EXAMPLE 1

### VAc/sulfonate 90:10

Initial charge: 120 g of methanol, 360 g of vinyl acetate
Feed 1: 156.9 g of sodium vinylsulfonate (25% by weight solution in water)
Feed 2: 1.2 g of tert-butyl perneodecanoate, 50 g of methanol

Preparation took place in a 2 liter stirred vessel under a nitrogen atmosphere. The initial charge was heated to 65° C. at a stirrer speed of 100 rpm. Then feed 1 was added over a period of 2 hours and feed 2 was added over a period of 3 hours. Polymerization was then continued for 2 hours. The organic solvent was then removed by steam distillation, and a cloudy aqueous solution with a solids content of 25.4% by weight was obtained. The K value of the polymer was 25.5 (1% by weight solution in water).

### EXAMPLE 2

### VAc/sulfonate 90:10

Initial charge: 153.6 g of methanol, 360 g of vinyl acetate, 160 g of sodium allyl-sulfonate (25% by weight solution in water), 1.37 g of 2,2′-azobis(isobutyronitrile).

Preparation took place in a 2 liter stirred vessel under a nitrogen atmosphere. The initial charge was heated to 65° C. at a stirrer speed of 100 rpm. Polymerization was then carried out at 65° C. for 8 hours and subsequently the organic solvent was removed by steam distillation and the aqueous solution was adjusted to a solids content of 32% by weight. The K value of the polymer was 5.9 (1% by weight solution in water).

### EXAMPLE 3

### VAc/sulfonate 80:20

Initial charge: 150 g of methanol, 160 g of vinyl acetate, 160 g of sodium allylsulfonate (25% by weight solution in water), 0.69 g of 2,2′-azobis(isobutyronitrile).

Preparation took place in analogy to example 2. The polymer was isolated after the steam distillation by freeze drying. The K value was 7.6 (1% by weight solution in water).

### EXAMPLE 4

### VAc/sulfonate 90:10

Initial charge: 300 g of methanol, 180 g of vinyl acetate
Feed 1: 250 g of methanol, 20 g of potassium sulfopropyl methacrylate (25% by weight solution in water)
Feed 2: 1.5 g of tert-butyl perneodecanoate, 50 g of methanol
Feed 3: 1.5 g of tert-butyl perneodecanoate

Preparation took place in a 2 liter stirred vessel under a nitrogen atmosphere. The initial charge was heated to an internal temperature of 64° C. at a stirrer speed of 100 rpm. Then feed 1 was added over a period of 4 hours and feed 2 was added over a period of 5 hours. Subsequently, feed 3 was added polymerization was continued at 64° C. for 2 hours. The organic solvent was then removed by steam distillation, and a cloudy white aqueous solution with a solids content of 27.8% by weight was obtained.

### EXAMPLE 5

Initial charge: 120 g of 1-propanol
Feed 1: 114.3 g of sodium allylsulfonate (35% by weight in water)
3.40 g of sulfuric acid
   Feed 2: 1.20 g of 2,2′-azobis(methyl isobutyrate)
50 g of 1-propanol
   Feed 3: 360 g of vinyl acetate
   Feed 4: 4.00 g of 2,2′-azobis(2-amidinopropane) dihydrochloride
50 g of water

Preparation took place in a 2 liter stirred vessel under a nitrogen atmosphere. The initial charge was heated to an internal temperature of 72° C. at a stirrer speed of 100 rpm. Then feed 1 and feed 3 were added over a period of 3 hours and feed 2 was added over a period of 4 hours. Subsequently, polymerization was continued at 72° C. for 1 hour. Then feed 4 was added and polymerization was continued at 72° C. for a further 2 hours. Subsequently, the organic solvent was removed by steam distillation, and a cloudy white aqueous solution with a solids content of 26% by weight was obtained. The K value of the polymer was 9.4 (1% by weight solution in water).

### EXAMPLE 6

Example 6 was prepared in analogy to example 5 except that the sodium allylsulfonate solution used was not neutralized with sulfuric acid. The resulting aqueous solution has a solids content of 22.1%, and the K value of the polymer was 9.1 (1% by weight solution in water).

### Preparation of Solubilizates

2 g of the copolymer were weighed into a glass beaker. A drug was then weighed into each mixture in order to obtain a supersaturated solution, as follows. (If the mass weighed in dissolved in the medium, the weight was increased until a sediment formed.)

Amount of active ingredient weighed in: 17-β-estradiol 0.2 g; piroxicam 0.2; clotrimazole 0.2 g; carbamazepine 0.3 g; ketoconazole 0.25 g; griseofulvin 0.25 g; cinnarizine 0.25 g.

Phosphate buffer of pH 7.0 was then added until solubilizer and phosphate buffer were present in the ratio of 1:10 by weight. A magnetic stirrer was used to stir this mixture at 20° C. for 72 hours. A resting period of at least 1 hour followed. The mixture was filtered and then measured by photometry, and the content of active ingredient was determined.

| | Copolymer of Ex. No./ | | | | |
|---|---|---|---|---|---|
| | Solubilization at 20° C. in [g/100 ml] | | | | |
| Carbamazepine | 1/0.13 | 2/0.16 | 3/0.12 | 4/0.15 | 5/0.15 |

| | Copolymer of Ex. No./ | | | | | |
|---|---|---|---|---|---|---|
| | Solubilization at 20° C. in [g/100 ml] | | | | | |
| Estradiol | 1/0.09 | 2/0.07 | 3/0.04 | 4/0.06 | 5/0.12 | 6/0.11 |

| | Copolymer of Ex. No./ | | | | | |
|---|---|---|---|---|---|---|
| | Solubilization at 20° C. in [g/100 ml] | | | | | |
| Piroxicam | 1/0.30 | 2/0.21 | 3/0.18 | 4/0.19 | 5/0.22 | 6/0.20 |

| | Copolymer of Ex. No./ | | | | | |
|---|---|---|---|---|---|---|
| | Solubilization at 20° C. in [g/100 ml] | | | | | |
| Clotrimazole | 1/0.08 | 2/0.17 | 3/0.08 | 4/0.12 | 5/0.17 | 6/0.16 |

### Preparation of Solid Solutions: General Procedure

The polymer-active ingredient mixture was produced by weighing the active ingredient and the polymer in the ratio 1:1 by weight into a suitable glass vessel (2 g of each) and then adding 16 ml of dimethylformamide as solvent. The mixture was stirred with a magnetic stirrer at 20° C. for 24 hours. The solution was then applied to a glass plate using a 120 μm knife. This plate was dried under a hood at RT for 0.5 hour and then dried in a drying oven at 50° C. and 10 mbar for a further 0.5 hour in order to remove the solvent quantitatively. The samples were subsequently inspected visually. If the films were clear and the active ingredient did not crystallize after 7 days, the active ingredient was assessed as stably dissolved in the polymer (indication in Table 1: 50% dissolved). If no solid solution could be obtained with an active ingredient content of 50% by weight, the experiment was repeated with an active ingredient loading of 33% by weight (indicated in table: 33% dissolved). The copolymers of the invention showed overall a greater capacity to form a solid solution.

TABLE
Stability of a solid solution

| Copolymer of | Carbamazepine | Estradiol | Piroxicam | Clotrimazole |
|---|---|---|---|---|
| Ex. 1 | 33% dissolved | — | — | 33% dissolved |
| Ex. 2 | 33% dissolved | — | — | 50% dissolved |
| Ex. 4 | 33% dissolved | 33% dissolved | 33% dissolved | 33% dissolved |
| Ex. 5 | 33% dissolved | 33% dissolved | 33% disssolved | 33% dissolved |
| Ex. 6 | 33% dissolved | 33% dissolved | 33% dissolved | 33% dissolved |

1.-16. (canceled)

17. A preparation of slightly water-soluble substances comprising as solubilizers copolymers obtainable by free radical-initiated copolymerization of
a) 85 to 98% by weight of vinyl acetate; and
b) 2 to 15% by weight of at least one monoolefinically unsaturated monomer having sulfonate groups.

18. The preparation according to claim 17, in which the slightly water-soluble substance is present in the copolymers in the form of a solid solution.

19. The preparation according to claim 17, comprising a bioactive substance as slightly water-soluble substance.

20. The preparation according to claim 17, comprising an active pharmaceutical ingredient as slightly water-soluble bioactive substance.

21. The preparation according to claim 20 in the form of a dosage form which can be administered orally.

22. The preparation according to claim 17, comprising a cosmetic active ingredient as slightly water-soluble bioactive substance.

23. The preparation according to claim 17, comprising an agrochemical active ingredient as slightly water-soluble bioactive substance.

24. The preparation according to claim 17, comprising a dietary supplement or a dietetic active ingredient as slightly water-soluble bioactive substance.

25. The preparation according to claim 17, comprising a colorant as a slightly water-soluble substance.

26. A process for preparing copolymers of vinyl acetate and monomers having sulfonate groups by free radical-initiated copolymerization, wherein the polymerization is initially carried out in the presence of a slightly water-soluble free-radical initiator, and then an after-polymerization is carried out in the presence of a water-soluble free-radical initiator.

27. A copolymer obtainable by free radical-initiated copolymerization of
a) 85 to 98% by weight of vinyl acetate; and
b) 2 to 15% by weight of at least one sulfoalkyl ester of acrylic acid or of methacrylic acid.

28. The copolymer according to claim 27, comprising sulfopropyl esters of acrylic acid or methacrylic acid as monomers b).

29. The copolymer according to claim 27, where monomers b) are employed in the form of their potassium salts.
